(19) United States
(12) Patent Application Publication
(10) Pub. No.: US 2021/0074386 A1
(43) Pub. Date: Mar. 11, 2021

(54) DISSIPATIVE PARTICLE DYNAMICS METHOD FOR SIMULATING INTERFACIAL POLYMERIZATION PROCESS OF HYDROGEL MEMBRANE

(71) Applicant: Energy Research Institute of Jiangxi Academy of Sciences, Nanchang (CN)

(72) Inventors: Zhaohuan MAI, Nanchang (CN); Cheng JIANG, Nanchang (CN); Shushan YUAN, Nanchang (CN); Jihai XIONG, Nanchang (CN); Jiujiu WU, Nanchang (CN); Shuanglin GUI, Nanchang (CN); Miaohui ZHANG, Nanchang (CN)

(21) Appl. No.: 16/827,488

(22) Filed: Mar. 23, 2020

(30) Foreign Application Priority Data
Sep. 11, 2019 (CN) 201910859079.6

(51) Int. Cl. G16C 10/00; C08J 5/00; C08J 3/075

(52) U.S. Cl. CPC G16C 10/00; C08J 2377/00; C08J 3/075; C08J 5/00

(57) ABSTRACT

The present invention relates to a dissipative particle dynamics (DPD) method for simulating an interfacial polymerization process of a hydrogel membrane. The method includes the following steps: 1) selecting various components with chemical structures thereof in a hydrogel phase and an organic phase of an interfacial polymerization system; 2) constructing a DPD model of solvent, hydrogel membrane material and water-soluble monomer in the hydrogel phase and a DPD model of organic solvent and oil-soluble monomer in the organic phase; 3) establishing a DPD model of the interfacial polymerization system composed of the hydrogel phase and the organic phase; 4) calculating an interaction parameter between DPD beads, that is, a conservative force parameter; 5) performing a DPD simulation by using Materials Studio software; and 6) evaluating an influencing factor determining the performance of the separation layer during the interfacial polymerization process according to the calculation file.

### TECHNICAL FIELD

The present invention belongs to the field of high-performance membrane materials, and in particular, relates to a dissipative particle dynamics (DPD) method for simulating an interfacial polymerization process of a hydrogel membrane by using a computer.

### BACKGROUND

With the shortage of water resources and the increasingly serious pollution of water, membrane separation technology, as one of the economic and efficient technologies for sewage treatment, seawater desalination and brackish water desalination, has broad market applications. Membrane materials, as the core of membrane separation technology, directly affect the separation performance of membranes and the application of membrane technology. The preparation of high-performance membrane materials is a hotspot for continuous development and research in the industrial and academic areas. At present, commercial reverse osmosis (RO) membranes, nanofiltration (NF) membranes and organic solvent NF membranes are generally prepared through the interfacial polymerization of an amine monomer in an aqueous phase and a polyacyl chloride monomer in an organic phase. The water-soluble monomer and the polyacyl chloride monomer form a selective layer (polyamide) on the surface of a substrate. In the process of interfacial polymerization, the concentration of the monomers, the reaction time and the structure of the substrate are the key factors affecting the performance of the finally prepared polyamide thin-film composite (PA-TFC) membrane.

Recently, researchers have added polymers (such as Kevlar fiber), instead of a conventional ultrafiltration (UF) membrane as a substrate into a reaction solution to prepare a superior ultra-thin PA-TFC membrane. The performance of a PA-TFC membrane prepared by interfacial polymerization with a hydrogel membrane surpasses that of a PA-TFC membrane using a conventional UF substrate. Therefore, the development and application of hydrogel composite membranes has become a research hotspot. Great progress has been made in the synthesis and modification of membrane materials. However, there is still insufficient research and explanation on the microstructural properties and mechanism of interfacial polymerization for the preparation of high-performance membrane materials. Therefore, it is important to research the microstructure characteristics and mechanism of interfacial polymerization for the preparation of high-performance composite membranes.

At present, widely used experimental characterization and detection methods include scanning electron microscope (SEM), transmission electron microscope (TEM) and atomic force microscope (AFM). They are difficult to meet requirements for the quantitative analysis of surface microscopic characteristics and dynamic changes of water-soluble monomers in hydrogel membrane systems and in interfacial polymerization at an atomic or molecular level. It is also difficult to explain the interfacial polymerization mechanism at the atomic or molecular level. Therefore, it is still a huge challenge in experiments to precisely control the structure and performance of a PA-TFC membrane prepared by the interfacial polymerization of a hydrogel membrane. Compared with experimental methods, computer simulation can help researchers understand the interfacial polymerization process and clarify possible influence of monomer/solvent concentration, structure and chemical properties, etc. on the polymerization from a microscopic/mesoscopic perspective. It is a powerful means to research the interfacial polymerization mechanism of separation membranes. Dissipative particle dynamics (DPD) simulation technology makes up for the lack of experimental methods. It provides a new idea for the in-depth research of the thermodynamic and dynamic influencing factors of the interfacial polymerization of the active layer in the PA-TFC membranes. It also provides a reliable theoretical basis for exploring the interfacial polymerization mechanism and further improving the interfacial polymerization process to obtain high-performance membrane materials.

### SUMMARY

In view of the shortcomings of the prior art, the present invention provides a method for using the software Materials Studio and dissipative particle dynamics (DPD) simulation to research the influencing factors on the thermodynamic and dynamic properties of a hydrogel assisted PA-TFC membrane during the interfacial polymerization process from a mesoscopic perspective. The present invention expands the understanding of the interfacial polymerization mechanism of a high-performance separation membrane.

The objective of the present invention is achieved by the following technical solutions.

A DPD method for simulating an interfacial polymerization process of a hydrogel membrane includes the following steps:

step 1: selecting various components with chemical structures thereof in a hydrogel phase and an organic phase of an interfacial polymerization system, where the hydrogel phase includes a solvent, a hydrogel membrane material and a water-soluble monomer; the organic phase includes an organic solvent and an oil-soluble monomer;

step 2: constructing a DPD model of solvent, hydrogel membrane material and water-soluble monomer in the hydrogel phase and a DPD model of organic solvent and oil-soluble monomer in the organic phase;

step 3: establishing a DPD structure model of the interfacial polymerization system composed of the hydrogel phase and the organic phase;

step 4: calculating an interaction parameter between DPD beads, that is, a conservative force parameter;

step 5: performing a DPD simulation by using Materials Studio software, and obtaining a trajectory file and a related calculation file of each DPD bead after system equilibrium; and

step 6: observing a structural characteristic of a hydrogel assisted PA-TFC membrane generated by the interfacial polymerization, according to a simulation result of step 5, and analyzing an influencing factor determining the performance of the separation layer during the interfacial polymerization process according to the calculation file.

Further, in the hydrogel phase, the solvent is water, and the hydrogel membrane material is one of polyparaphenylene terephthalamide, chitosan, cellulose, sodium alginate or polyvinyl alcohol.

Further, the water-soluble monomer is one of piperazine, 2-methylpiperazine, 2,5-dimethylpiperazine, 4-aminomethylpiperazine, 2,5-diethylpiperazine, α-cyclodextrin, β-cyclodextrin, γ-cyclodextrin, δ-cyclodextrin, p-phenylenediamine, m-phenylenediamine, mesitylenetriamine, diaminotoluene, ethylenediamine, propanediamine, phenyldimethyldiamine, 1,3-diaminocyclohexane or 1,4-diaminocyclohexane; the water-soluble monomer has a concentration of 0.01-8.0 wt %.

Further, the water-soluble monomer is preferably piperazine, m-phenylenediamine or cyclodextrin.

Further, the organic solvent in the organic phase is one or more of n-hexane, cyclohexane, heptane, octane, naphtha, Isopar-E, Isopar-G, Isopar-L or mineral oil; the oil-soluble monomer in the organic phase is a polyacyl chloride monomer, which is one of trimesoyl chloride, terephthaloyl chloride, isophthaloyl chloride, terephthaloyl chloride, benzenetrisulfonyl chloride, propyl triacyl chloride, butyl triacyl chloride, pentyl triacyl chloride, glutaryl chloride, adipoyl chloride, maleic diacyl chloride, cyclopropane triacyl chloride, cyclobutane triacyl chloride, cyclobutane tetraacyl chloride, cyclopentane diacyl chloride, cyclopentane triacyl chloride, cyclopentane tetraacyl chloride, cyclohexane diacyl chloride, cyclohexane triacyl chloride or cyclohexane tetraacyl chloride; the oil-soluble monomer has a concentration of 0.01-4.0 wt %.

Further, step 2 is specifically as follows:

(1) coarsely graining each substance in the system and defining different types of DPD beads, according to a chemical structure of the aqueous solvent, the hydrogel membrane material and the monomer in the hydrogel phase and the organic solvent as well as the oil-soluble monomer in the organic phase; and

(2) setting a bead type by using Materials Visualizer module of Materials Studio software, and using a corresponding DPD bead to construct a DPD model for a solvent molecule, a hydrogel membrane material molecule, a water-soluble monomer molecule, an organic solvent molecule and an oil-soluble monomer molecule.

Further, step 3 is specifically as follows:

(1) constructing a cube box by using Materials Studio software, and dividing the box into upper and lower layers evenly, where the upper layer is set as an organic phase for placing the organic solvent and the oil-soluble monomer during the interfacial polymerization process, and the lower layer is set as the hydrogel phase for placing the solvent molecule, the hydrogel membrane material molecule and the water-soluble monomer during the interfacial polymerization process; and

(2) determining a number of the solvent molecule, the hydrogel membrane material molecule, the water-soluble monomer molecule, the organic solvent molecule and the oil-soluble monomer molecule by a monomer concentration required for the interfacial polymerization.

Further, step 4 is specifically: obtaining a Flory-Huggins parameter between each pair of DPD beads by a molecular dynamics simulation or through a reference, and then calculating an interaction parameter between each pair of DPD beads according to a DPD theory.

Further, step 5 is specifically:

(1) optimizing the structure of the constructed interfacial polymerization system by Geometry Optimization in Mesocite, and after optimization, fixing a position of the hydrogel membrane material molecule in a solvent phase of the interfacial polymerization system;

(2) performing a DPD simulation of the system by Mesocite module of Materials Studio software by using the DPD model of the interfacial polymerization system obtained by the construction method in step 3 and the conservative force parameter obtained in step 4, to obtain an interfacial polymerization structure in equilibrium; and

(3) outputting and saving a trajectory file and a related calculation file of each bead in the DPD simulation, where the related file includes an interaction energy file, a concentration file, a density file, a radial distribution function file, a mean square displacement file and a mutual distance file.

Further, step 6 is specifically as follows:

(1) outputting a structure when the DPD model of the hydrogel membrane interfacial polymerization system obtained in step 5 reaches a stable equilibrium state, and observing a trajectory of all DPD beads;

(2) drawing a trajectory evolution map (snapshot) to reflect a structure of a polymer layer formed by the interfacial polymerization of the two monomers over time, according to a trajectory file and a related calculation file of water-soluble monomer and oil-soluble monomer beads that react with each other; calculating an evolution map of the concentration of the water-soluble monomer and the oil-soluble monomer in an interfacial polymerization layer over time, and examining the concentration distribution of the two monomers near the interfacial polymerization layer at different time; analyzing an influence of a gel material on the water-soluble monomer and an interfacial polymerization rate thereof according to a trajectory of the water-soluble monomer and the hydrogel membrane material molecule; and

(3) obtaining through the above analysis a factor determining the structure and performance of a polymer during the interfacial polymerization process and an influence law thereof.

The present invention aims at the interfacial polymerization process of the hydrogel assisted PA-TFC membrane. The present invention combines a DPD simulation and an experiment to research the influence of various factors on the structure and performance of the hydrogel assisted PA-TFC membrane. The present invention explores the reaction mechanism of the interfacial polymerization process from a microscopic perspective and lays a theoretical foundation for improving the interfacial polymerization process to obtain a high-performance membrane material.

The present invention uses a DPD simulation method to research the interfacial polymerization mechanism of the hydrogel assisted PA-TFC membrane. Compared with a traditional method, the present invention has the following significant advantages. (1) The present invention researches the structure and performance of the hydrogel assisted PA-TFC membrane formed by interfacial polymerization and an influencing mechanism of various factors during the interfacial polymerization process from a mesoscopic perspective. (2) The present invention provides a dynamic visualization effect of the interfacial polymerization, which is conducive to further understanding of the interfacial polymerization mechanism. (3) The research makes up for the lack of experimental means, and makes it possible to visually observe the formation process of the hydrogel assisted PA-TFC membrane and an influencing factor on the structure and performance thereof from a microscopic perspective. (4) This method applies to chemical, environmental and life sciences fields related to membrane material preparation and water treatment, etc.

### BRIEF DESCRIPTION OF DRAWINGS

FIG. 1 is a diagram of the coarse graining with dissipative particle dynamics (DPD) method of the solvent, the gel membrane material and the water-soluble monomer in the hydrogel phase and the organic solvent, the oil-soluble monomer in the organic phase during an interfacial polymerization process in Embodiment 2.

FIG. 2 is the initial distribution diagram of various components in the hydrogel phase and the organic phase for the interfacial polymerization in Embodiment 2.

FIG. 3 is a snapshot of a PA layer formed by piperazine (PIP) in the hydrogel phase and trimesoyl chloride (TMC) in the organic phase through interfacial polymerization in equilibrium state in Embodiment 2.

### DETAILED DESCRIPTION

To better understand the present invention, the technical solution of the present invention is described in further detail below with reference to specific implementations, but the present invention is not limited thereto. Any modification or equivalent replacement of the technical solution of the present invention made without departing from the spirit and scope of the technical solution of the present invention should fall within the protection scope of the present invention.

### Embodiment 1

This embodiment uses Materials Studio software and a dissipative particle dynamics (DPD) simulation method to simulate an interfacial polymerization process of a hydrogel assisted PA-TFC membrane and explore a reaction mechanism on a calculation server, including the following aspects:

step 1: determine various components and a chemical structure thereof in a hydrogel phase and an organic phase of an interfacial polymerization system, where the hydrogel phase includes a solvent, a hydrogel membrane material and a water-soluble monomer; the organic phase includes an organic solvent and an oil-soluble monomer;

step 2: construct a DPD model of solvent, hydrogel membrane material and water-soluble monomer in the hydrogel phase and a DPD model of organic solvent and oil-soluble monomer in the organic phase;

step 3: establish a DPD structure model of the interfacial polymerization system composed of the hydrogel phase and the organic phase;

step 4: calculate an interaction parameter between DPD beads, that is, a conservative force parameter;

step 5: perform a DPD simulation by using Materials Studio software, and obtain a trajectory file and a related calculation file of each DPD bead after system equilibrium; and

step 6: observe a structural characteristic of a hydrogel assisted PA-TFC membrane generated by the interfacial polymerization, according to a simulation result of step 5, and research an influencing factor determining the performance of the separation layer during the interfacial polymerization process according to the calculation file.

Specific steps are as follows:

(1) determine various components and a chemical structure thereof in a hydrogel phase and an organic phase of an interfacial polymerization system, where the hydrogel phase includes a solvent, a hydrogel membrane material and a water-soluble monomer; the organic phase includes an organic solvent and an oil-soluble monomer; different interfacial polymerization monomers and hydrogel membrane materials form hydrogel assisted PA-TFC membranes with different structures and properties, and appropriate solvents, hydrogel membrane materials and monomers are selected according to an actual situation;

(2) coarsely grain each substance in the system and define different types of DPD beads, according to a chemical structure of the aqueous solvent, the hydrogel membrane material and the monomer in the water phase and the organic phase;

(3) set a bead type by using Materials Visualizer module of Materials Studio software, and use a corresponding DPD bead to construct a DPD model for a water molecule, a hydrogel membrane material molecule, a water-soluble monomer molecule, an organic solvent molecule and an oil-soluble monomer molecule;

(4) construct a cube box by using Materials Studio software, and divide the box into upper and lower layers evenly, where the upper layer is set as an organic phase for placing the organic solvent and the oil-soluble monomer during the interfacial polymerization process, and the lower layer is set as the hydrogel phase for placing the water molecule, the hydrogel membrane material molecule and the water-soluble monomer during the interfacial polymerization process; a number of the solvent molecule, the hydrogel membrane material molecule, the water-soluble monomer molecule, the organic solvent molecule and the oil-soluble monomer molecule is determined by a concentration required for the interfacial polymerization;

(5) obtain a Flory-Huggins parameter between each pair of DPD beads by a molecular dynamics simulation or through a reference, and then calculate an interaction (conservative force) parameter between each pair of DPD beads according to a DPD theory, where a relationship between the Flory-Huggins parameter and the DPD conservative force parameter is described in Formulas 1 and 2:

$$\begin{matrix}{a}_{i i} = \frac{( 16 {N}_{m} - 1 ) {k}_{B} T}{0.2 ρ} & ( 1 ) \\ {a}_{i j} = {a}_{i i} + \frac{{χ}_{ij}}{0.231} & ( 2 )\end{matrix}$$

where aᵢᵢ represents the interaction parameter between the same DPD beads;

aᵢⱼ represents the interaction parameter between different DPD beads;

Nₘ represents the coarse graining level in the DPD simulation, that is, the number of water molecules included in one DPD bead;

k_{B}T represents the energy unit in the DPD simulation;

ρ represents the density of the DPD simulation system; in this embodiment, ρ=3;

χᵢⱼ represents the Flory-Huggins parameter between different DPD beads, which is obtained through molecular dynamics simulation or from a credible scientific reference;

(6) optimize the structure of the constructed interfacial polymerization system by Geometry Optimization in Mesocite, where an optimization condition includes a self-calculated DPD force field, an optimization level “Customized”, an electrostatic interaction “Ewald”, a Van der Waals' force (VDW) “bead based” and a cutoff distance 12.5 Å; and after optimization, fix a position of the hydrogel membrane material molecule in the water phase of the interfacial polymerization system;

(7) perform a DPD simulation of the system by Mesocite module of Materials Studio software by using the DPD model of the interfacial polymerization system obtained by the construction method in step (4) and the conservative force parameter obtained in step (5), to obtain an interfacial polymerization structure in equilibrium, where a force field used in the DPD simulation is the same as that in step (6), and both use a conservative force parameter calculated according to Formulas 1 and 2; a simulation time and a step size are adjusted based on an actual situation and a size of the simulation system;

(8) output and save a trajectory file and a related calculation file of each bead in the DPD simulation, where the related file includes an interaction energy file, a concentration file, a density file, a radial distribution function file, a mean square displacement file and a mutual distance file;

(9) output a structure when the DPD model of the hydrogel membrane interfacial polymerization system obtained in step (7) reaches a stable equilibrium state, and observe a trajectory of all DPD beads;

(10) draw a trajectory evolution map (snapshot) to reflect a structure of a polymer layer formed by the interfacial polymerization of the two monomers over time, according to a trajectory file and a related calculation file of water-soluble monomer and oil-soluble monomer beads that react with each other; calculate an evolution map of the concentration of the water-soluble monomer and the oil-soluble monomer in an interfacial polymerization layer over time, and examine the concentration distribution of the two monomers near the interfacial polymerization layer at different time; analyze an influence of a gel material on the water-soluble monomer and an interfacial polymerization rate thereof according to a trajectory of the water-soluble monomer and the hydrogel membrane material molecule; and

(11) analyze through the above analysis a factor determining the structure and performance of a polymer during the interfacial polymerization process and an influence law thereof.

### Embodiment 2

This embodiment uses a polyparaphenylene terephthalamide (PPTA, also known as Kevlar) to prepare a hydrogel membrane. For example, a water-soluble piperazine (PIP) monomer and a trimesoyl chloride (TMC) monomer in n-hexane (Hexane) are used for interfacial polymerization to form a composite membrane separation layer, specifically as follows:

(1) determine various components and a chemical structure thereof in a hydrogel phase and an organic phase of an interfacial polymerization system, where water (H₂O), the PPTA and the PIP respectively serve as a solvent, a hydrogel membrane material and a water-soluble monomer in the hydrogel phase; the Hexane and the TMC respectively serve as an organic solvent and an oil-soluble monomer in the organic phase;

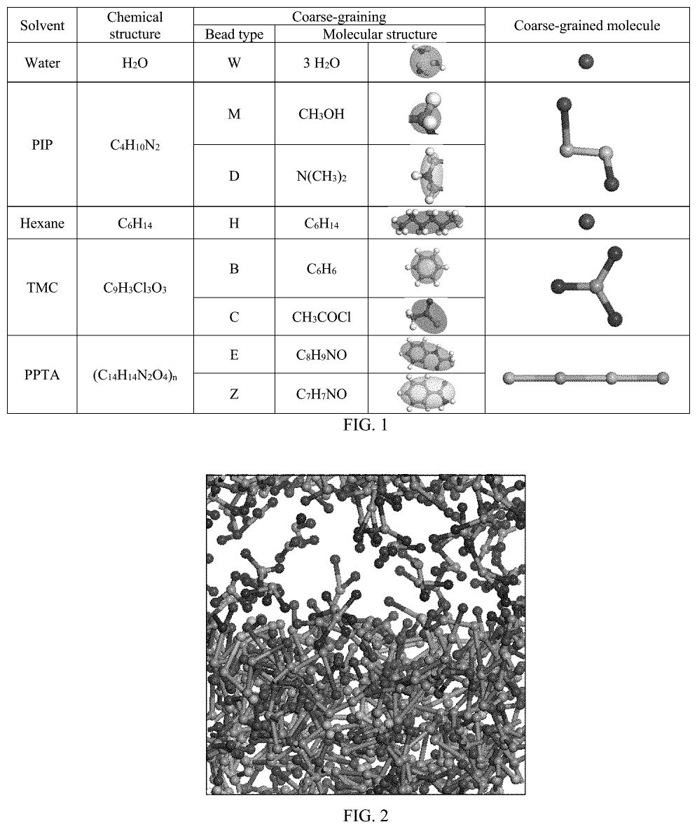

(2) coarsely grain each substance in the system and define different types of DPD beads, as shown in “Bead type” in FIG. 1, according to a molecular structure of the water, the PPTA, the PIP, the Hexane and the TMC;

(3) set a bead type by using Materials Visualizer module of Materials Studio software, and use a corresponding DPD bead to construct a DPD model for a water molecule, a PIP molecule an Hexane molecule and a TMC molecule, as shown in “Coarse-grained molecule” in FIG. 1;

(4) construct a cube box with a volume of 100×100×100 Å³ by using Materials Studio software, and divide the box into upper and lower layers evenly (each layer having a volume of 100×100×50 Å³), where the upper layer is set as an organic phase for placing the Hexane and TMC molecules during the interfacial polymerization process, and the lower layer is set as the hydrogel phase for placing the water molecule, the PPTA molecule and the PIP molecule during the interfacial polymerization process; FIG. 2 shows a constructed interfacial polymerization system, where in the organic phase, the Hexane and the TMC have a ratio of 0.95:0.05, and in the hydrogel phase, the water, the PPTA and the PIP have a ratio of 0.85:0.05:0.1; in order to facilitate observation, the water molecule in the hydrogel phase and the Hexane molecule in the organic phase are set to be invisible;

(5) obtain a Flory-Huggins parameter between each pair of DPD beads by a molecular dynamics simulation or through a reference, and then calculate an interaction (conservative force) parameter between each pair of DPD beads according to a DPD theory, where a relationship between the Flory-Huggins parameter and the DPD conservative force parameter (the same as that in Embodiment 1) is described in Formulas 1 and 2;

(6) optimize the structure of the constructed interfacial polymerization system by Geometry Optimization in Mesocite, where an optimization condition includes a self-calculated DPD force field, an optimization level “Customized”, an electrostatic interaction “Ewald”, a Van der Waals' force (VDW) “bead based” and a cutoff distance 12.5 Å; and after optimization, fix a position of the hydrogel membrane material molecule in the water phase of the interfacial polymerization system;

(7) perform a DPD simulation of 500,000 steps (about 75 ns) of the system after structure optimization by Mesocite module of Materials Studio software to obtain an interfacial polymerization structure in equilibrium, where a force field used in the DPD simulation is the same as that in step (6), both using a conservative force parameter calculated according to Formulas 1 and 2; the trajectories of all DPD beads are saved in one frame every 500 steps;

(8) output and save a trajectory file and a related calculation file of the PIP molecule and the TMC molecule in the DPD simulation after an equilibrium state is reached, where the related file includes an interaction energy file, a concentration file, a density file, a radial distribution function file, a mean square displacement file and a mutual distance file;

(9) output a structure when the DPD model of the interfacial polymerization system obtained in step (7) reaches a stable equilibrium state, and observe a trajectory of all DPD beads;

(10) draw a trajectory evolution map (snapshot) to reflect a structure of a polymer layer formed by the interfacial polymerization of the two monomers over time, according to a trajectory file and a related calculation file of the DPD molecules of the PIP and TMC monomers that are subject to the interfacial polymerization; calculate an evolution map of the concentration of the PIP and TMC in an interfacial polymerization layer over time, and examine the concentration distribution of the two monomers near the interfacial polymerization layer at different time; analyze an influence of the PPTA on the PIP and an interfacial polymerization rate thereof according to the trajectory of the PIP and the PPTA; and

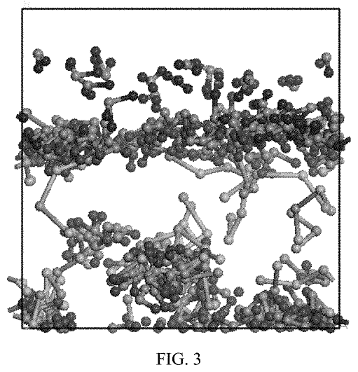

(11) form a composite membrane separation layer by the PIP in the hydrogel phase and the TMC in the organic phase through interfacial polymerization in an equilibrium state, as shown in FIG. 3 (a snapshot). The above analysis shows that the PPTA in the hydrogel phase has a strong adsorption effect on the PIP molecule. During diffusion in the hydrogel phase, one part of the PIP molecule is adsorbed on a PPTA chain while the other part continues to diffuse to the interfacial polymerization layer to participate in the interfacial polymerization. During the interfacial polymerization, a polyamide polymer membrane is formed through the interfacial polymerization of a bead D in the PIP molecule and a bead C in the TMC molecule. The concentration of the PPTA, PIP and TMC monomers have an important influence on the thickness, pore size and porosity of the polymer membrane formed.

The above describes the preferred implementations of the present patent in detail, but the present patent is not limited thereto. A person of ordinary skill in the art may make various changes without departing from the spirit of the present patent.

What is claimed is:

1. A dissipative particle dynamics (DPD) method for simulating an interfacial polymerization process of a hydrogel membrane, wherein the method comprises the following steps:
step 1: selecting various components with chemical structures thereof in a hydrogel phase and an organic phase of an interfacial polymerization system, wherein the hydrogel phase comprises a solvent, a hydrogel membrane material and a water-soluble monomer; the organic phase comprises an organic solvent and an oil-soluble monomer;
step 2: constructing a DPD model of solvent, hydrogel membrane material and water-soluble monomer in the hydrogel phase and a DPD model of organic solvent and oil-soluble monomer in the organic phase;
step 3: establishing a DPD structure model of the interfacial polymerization system composed of the hydrogel phase and the organic phase;
step 4: calculating an interaction parameter between DPD beads, that is, a conservative force parameter;
step 5: performing a DPD simulation by using Materials Studio software, and obtaining a trajectory file and a related calculation file of each DPD bead after system equilibrium; and
step 6: observing a structural characteristic of a hydrogel assisted PA-TFC membrane generated by the interfacial polymerization, according to a simulation result of step 5, and analyzing an influencing factor determining the performance of the separation layer during the interfacial polymerization process according to the calculation file.

2. The DPD method for simulating an interfacial polymerization process of a hydrogel membrane according to claim 1, wherein in the hydrogel phase, the solvent is water, and the hydrogel membrane material is one of polyparaphenylene terephthalamide, chitosan, cellulose, sodium alginate or polyvinyl alcohol.

3. The DPD method for simulating an interfacial polymerization process of a hydrogel membrane according to claim 1, wherein the water-soluble monomer is one of piperazine, 2-methylpiperazine, 2,5-dimethylpiperazine, 4-aminomethylpiperazine, 2,5-diethylpiperazine, α-cyclodextrin, β-cyclodextrin, γ-cyclodextrin, δ-cyclodextrin, p-phenylenediamine, m-phenylenediamine, mesitylenetriamine, diaminotoluene, ethylenediamine, propanediamine, phenyldimethyldiamine, 1,3-diaminocyclohexane or 1,4-diaminocyclohexane; the water-soluble monomer has a concentration of 0.01-8.0 wt %.

4. The DPD method for simulating an interfacial polymerization process of a hydrogel membrane according to claim 1, wherein the water-soluble monomer is preferably piperazine, m-phenylenediamine or cyclodextrin.

5. The DPD method for simulating an interfacial polymerization process of a hydrogel membrane according to claim 1, wherein the organic solvent in the organic phase is one or more of n-hexane, cyclohexane, heptane, octane, naphtha, Isopar-E, Isopar-G, Isopar-L or mineral oil; the oil-soluble monomer in the organic phase is a polyacyl chloride monomer, which is one of trimesoyl chloride, terephthaloyl chloride, isophthaloyl chloride, terephthaloyl chloride, benzenetrisulfonyl chloride, propyl triacyl chloride, butyl triacyl chloride, pentyl triacyl chloride, glutaryl chloride, adipoyl chloride, maleic diacyl chloride, cyclopropane triacyl chloride, cyclobutane triacyl chloride, cyclobutane tetraacyl chloride, cyclopentane diacyl chloride, cyclopentane triacyl chloride, cyclopentane tetraacyl chloride, cyclohexane diacyl chloride, cyclohexane triacyl chloride or cyclohexane tetraacyl chloride; the oil-soluble monomer has a concentration of 0.01-4.0 wt %.

6. The DPD method for simulating an interfacial polymerization process of a hydrogel membrane according to claim 1, wherein step 2 is specifically as follows:
(1) coarsely graining each substance in the system and defining different types of DPD beads, according to a chemical structure of the aqueous solvent, the hydrogel membrane material and the monomer in the hydrogel phase and the organic solvent as well as the oil-soluble monomer in the organic phase; and
(2) setting a bead type by using Materials Visualizer module of Materials Studio software, and using a corresponding DPD bead to construct a DPD model for a solvent molecule, a hydrogel membrane material molecule, a water-soluble monomer molecule, an organic solvent molecule and an oil-soluble monomer molecule.

7. The DPD method for simulating an interfacial polymerization process of a hydrogel membrane according to claim 1, wherein step 3 is specifically as follows:
(1) constructing a cube box by using Materials Studio software, and dividing the box into upper and lower layers evenly, wherein the upper layer is set as an organic phase for placing the organic solvent and the oil-soluble monomer during the interfacial polymerization process, and the lower layer is set as the hydrogel phase for placing the solvent molecule, the hydrogel membrane material molecule and the water-soluble monomer during the interfacial polymerization process; and
(2) determining a number of the solvent molecule, the hydrogel membrane material molecule, the water-soluble monomer molecule, the organic solvent molecule and the oil-soluble monomer molecule by a monomer concentration required for the interfacial polymerization.

8. The DPD method for simulating an interfacial polymerization process of a hydrogel membrane according to claim 1, wherein step 4 is specifically: obtaining a Flory-Huggins parameter between each pair of DPD beads by a molecular dynamics simulation or through a reference, and then calculating an interaction parameter between each pair of DPD beads according to a DPD theory.

9. The DPD method for simulating an interfacial polymerization process of a hydrogel membrane according to claim 1, wherein step 5 is specifically:
(1) optimizing the structure of the constructed interfacial polymerization system by Geometry Optimization in Mesocite, and after optimization, fixing a position of the hydrogel membrane material molecule in a solvent phase of the interfacial polymerization system;
(2) performing a DPD simulation of the system by Mesocite module of Materials Studio software by using the DPD model of the interfacial polymerization system obtained by the construction method in step 3 and the conservative force parameter obtained in step 4, to obtain an interfacial polymerization structure in equilibrium; and
(3) outputting and saving a trajectory file and a related calculation file of each bead in the DPD simulation, wherein the related file comprises an interaction energy file, a concentration file, a density file, a radial distribution function file, a mean square displacement file and a mutual distance file.

10. The DPD method for simulating an interfacial polymerization process of a hydrogel membrane according to claim 1, wherein step 6 is specifically as follows:
(1) outputting a structure when the DPD model of the hydrogel membrane interfacial polymerization system obtained in step 5 reaches a stable equilibrium state, and observing a trajectory of all DPD beads;
(2) drawing a trajectory evolution map (snapshot) to reflect a structure of a polymer layer formed by the interfacial polymerization of the two monomers over time, according to a trajectory file and a related calculation file of water-soluble monomer and oil-soluble monomer beads that react with each other; calculating an evolution map of the concentration of the water-soluble monomer and the oil-soluble monomer in an interfacial polymerization layer over time, and examining the concentration distribution of the two monomers near the interfacial polymerization layer at different time; analyzing an influence of a gel material on the water-soluble monomer and an interfacial polymerization rate thereof according to a trajectory of the water-soluble monomer and the hydrogel membrane material molecule; and
(3) obtaining through the above analysis a factor determining the structure and performance of a polymer during the interfacial polymerization process and an influence law thereof.
